(19) United States
(12) Patent Application Publication
(10) Pub. No.: US 2020/0309725 A1
(43) Pub. Date: Oct. 1, 2020

(54) PORTABLE MICROBIAL LOAD DETECTION

(71) Applicant: Pinnacle Bio, LLC, Orlando, FL (US)

(72) Inventors: Aatish Madhiwala, Burr Ridge, IL (US); Yogi A. Patel, Kennesaw, GA (US); Nilesh Mehta, Watchung, NJ (US)

(21) Appl. No.: 16/900,003

(22) Filed: Jun. 12, 2020

Related U.S. Application Data
(63) Continuation-in-part of application No. 15/834,926, filed on Dec. 7, 2017.

(51) Int. Cl. G01N 27/327; G01N 33/15; G01N 33/543; G01N 33/66; G01N 33/94

(52) U.S. Cl. CPC G01N 27/3274; G01N 33/15; G01N 33/9486; G01N 33/66; G01N 33/54366

(57) ABSTRACT

Portable microbial load detection is disclosed. For example, a test strip includes a calibration well with first, second, and third calibration traces and a sample well with first, second, and third test traces. A reader is configured to detect a connection with the test strip. A drive signal is applied to the test strip. A first voltage of the second calibration trace is measured indicating a reagent has been added to the calibration well. A second voltage of the third calibration trace is measured over a calibration time period and then used to calibrate the reader. An addition of a test sample in the sample well based is detected. A third voltage of the third test trace is measured over a test time period. A concentration of a detectable compound in the test sample is calculated and used to report a diagnosis state.

### CROSS-REFERENCES TO RELATED APPLICATIONS

This application is a continuation-in-part application of U.S. patent application Ser. No. 15/834,926, filed Dec. 7, 2017, which is hereby incorporated by reference in its entirety for all purposes.

### BACKGROUND

The present disclosure generally relates to a portable detection system of small molecule compounds as detectable compounds related to diseases caused by microbes, such as fungi, bacteria, and viruses. With advancements in microprocessors, battery technology, and electronic miniaturization, come possibilities for convenient, portable medical devices with capabilities previously reserved for large laboratory based equipment. Many diseases in humans as well as domesticated animals are caused by microbes, such as fungi, bacteria, and viruses. An accurate assessment of a concentration (e.g., a low concentration such as one below 1 mM) of infectious microbe in a given patient may be beneficial for both diagnosis and treatment of disease caused by such microbes. A fast and convenient test for such concentrations of infectious microbes in patient samples may improve treatment options and prognosis.

### SUMMARY

The present disclosure provides a new and innovative system, methods and apparatus for portable microbial load detection. In an example, a test strip includes a calibration well associated with first, second, and third calibration traces and a sample well associated with first, second, and third test traces. A reader includes an amplifier and a processor configured to execute to detect a connection with the test strip. A drive signal is applied to the first calibration trace and the first test trace. A first electrical signal (such as voltage) of the second calibration trace is measured indicating that a reagent has been added to the calibration well. A second electrical signal (such as voltage) of the third calibration trace is measured over a calibration time period associated with the reagent. The reader is calibrated to the reagent on the test strip based on the second electrical signal (such as voltage). An addition of a test sample in the sample well is detected based on measuring a third electrical signal (such as voltage) on the second test trace, where the first test sample includes the first reagent. A fourth electrical signal (such as voltage) of the third test trace is measured over a test time period. A concentration of a detectable compound in the test sample is calculated. A diagnosis state is reported based on the concentration of the detectable compound.

Additional features and advantages of the disclosed method and apparatus are described in, and will be apparent from, the following Detailed Description and the Figures.

In one aspect, the present disclosure provides a system of detecting a concentration of a detectable compound. The system comprises a first test strip including a calibration well and at least a first sample well, wherein the calibration well is associated with at least a first calibration trace, a second calibration trace, and a third calibration trace, and the first sample well is associated with at least a first test trace, a second test trace, and a third test trace, wherein at least one of the first sample well, the first test trace, the second test trace, and the third test trace comprises a precursor compound.

The system further comprises a reader including one or more processors and an amplifier, the one or more processors configured to execute to: detect a connection with the first test strip; apply a drive signal to the first test strip, wherein the drive signal is applied to the first calibration trace and the first test trace; measure a first electrical signal of the second calibration trace, wherein the first electrical signal indicates that a first reagent comprising a first concentration of the detectable compound has been added to the calibration well; measure a second electrical signal of the third calibration trace over a first calibration time period, wherein the third calibration trace is associated with the first reagent comprising the first concentration of the detectable compound; calibrate the reader to the first reagent on the first test strip based on the second electrical signal; detect an addition of a first test sample in the first sample well based on measuring a third electrical signal on the second test trace, wherein the first test sample comprises the first reagent, and wherein an incubation period elapses before the first test sample is added to the first sample well, and a first biological sample is added into the first reagent during the incubation period and the first biological sample comprises an enzyme, wherein the enzyme catalyzes a reaction of the precursor compound to produce a second concentration of the detectable compound; measure a fourth electrical signal of the third test trace over a first test time period;

calculate the second concentration of the detectable compound in the first test sample; and report a diagnosis state based on the concentration of the detectable compound.

In one embodiment, the electrical signal is voltage.

In one embodiment, the detectable compound is either glucose or paracetamol.

In one embodiment, insertion of the first test strip is based on detecting a transient electrical pulse after the insertion of the first test strip, and the first test strip is validated after detecting the transient electrical pulse.

In one embodiment, the one or more processors further executes to: plot the fourth electrical signal over the first test time period as a first electrical signal waveform; determine whether the first electrical signal waveform aligns with a control waveform associated with a medical diagnosis; responsive to determining that the first electrical signal waveform aligns with the control waveform, report the medical diagnosis; and responsive to determining that the first electrical signal waveform fails to align with the control waveform, display an error on the reader.

In one embodiment, the amplifier is a transimpedance amplifier.

In one embodiment, each of the first calibration trace, the second calibration trace, the third calibration trace, the first test trace, the second test trace, and the third test trace is connected to a separate channel in the transimpedance amplifier.

In one embodiment, at least two signals corresponding respectively to at least two of the first calibration trace, the second calibration trace, the third calibration trace, the first test trace, the second test trace, and the third test trace are multiplexed together in the transimpedance amplifier.

In one embodiment, the transimpedance amplifier in a first configuration mode measures a first signal type associated with a first tested medical condition associated with the first biological sample, and the transimpedance amplifier in a second configuration mode measures a second signal type associated with a second tested medical condition associated with a second biological sample.

In one embodiment, the reader is configured to recalibrate with a second test strip and a second reagent comprising a first concentration of the detectable compound on the second test strip, and after recalibrating, a second test sample comprising the second reagent is measured on the second test strip, wherein an incubation period elapses before the second test sample is added to a second sample well, and a second biological sample is added into the second reagent during the incubation period and the second biological sample comprises the enzyme, wherein the enzyme catalyzes the reaction of the precursor compound to produce a third concentration of the detectable compound.

In one embodiment, the second reagent is a separate aliquot of the first reagent.

In one embodiment, the first reagent and the second reagent are individually biologically isolated.

In one embodiment, the first test strip includes a second sample well biologically isolated from the first sample well, and the second sample well is used to measure a second test sample.

In one embodiment, the diagnosis state is reported in a graphical report, and the graphical report abstracts numerical measurements of the fourth electrical signal and the second concentration of the detectable compound.

In one embodiment, the detectable compound is paracetamol.

In one embodiment, the second concentration of the detectable compound is either 1 mM or below 1 mM.

In one embodiment, the second concentration of the detectable compound is a concentration selected from the group consisting of 0.9 mM, 0.8 mM, 0.7 mM, 0.6 mM, 0.5 mM, 0.4 mM, 0.5 mM, 0.4 mM, 0.3 mM, 0.2 Mm, 0.1 mM and 0.05 mM.

In another aspect, the present disclosure provides a method of detecting a concentration of a detectable compound. The method comprises detecting a connection with a test strip, wherein the test strip includes a calibration well associated with at least a first calibration trace, a second calibration trace, and a third calibration trace and at least a first sample well associated with at least a first test trace, a second test trace, and a third test trace, wherein at least one of the first sample well, the first test trace, the second test trace, and the third test trace comprises a precursor compound; applying a drive signal to the test strip, wherein the drive signal is applied to the first calibration trace and the first test trace; measuring a first electrical signal of the second calibration trace, wherein the first electrical signal indicates that a first reagent comprising a first concentration of the detectable compound has been added to the calibration well; measuring a second electrical signal of the third calibration trace over a first calibration time period, wherein the third calibration trace is associated with the first reagent comprising a first concentration of the detectable compound.

The method further comprises calibrating the reader to the first reagent on the first test strip based on the second electrical signal; detecting an addition of a first test sample in the first sample well based on measuring a third electrical signal on the second test trace, wherein the first test sample comprises the first reagent, and wherein an incubation period elapses before the first test sample is added to the first sample well, and a first biological sample is added into the first reagent during the incubation period and the first biological sample comprises an enzyme, wherein the enzyme catalyzes a reaction of the precursor compound to produce a second concentration of the detectable compound; measuring a fourth electrical signal of the third test trace over a first test time period; calculating a the second concentration of the detectable compound in the first test sample; and reporting a diagnosis state based on the concentration of the detectable compound.

In one embodiment, the electrical signal is voltage.

In one embodiment, the detectable compound is either glucose or paracetamol.

In one embodiment, the detectable compound is paracetamol.

In one embodiment, the second concentration of the detectable compound is 1 mM or below 1 mM.

In one embodiment, the second concentration of the detectable compound is a concentration selected from the group consisting of 0.9 mM, 0.8 mM, 0.7 mM, 0.6 mM, 0.5 mM, 0.4 mM, 0.5 mM, 0.4 mM, 0.3 mM, 0.2 Mm, 0.1 mM and 0.05 mM.

In another aspect, the present disclosure provides a non-transitory computer readable storage medium. The non-transitory computer readable storage medium stores one or more computer programs adapted to cause a processor based system to execute steps comprising: detecting a connection with a test strip, wherein the test strip includes a calibration well associated with at least a first calibration trace, a second calibration trace, and a third calibration trace and at least a first sample well associated with at least a first test trace, a second test trace, and a third test trace, wherein at least one of the first sample well, the first test trace, the second test trace, and the third test trace comprises a precursor compound; applying a drive signal to the test strip, wherein the drive signal is applied to the first calibration trace and the first test trace; measuring a first electrical signal of the second calibration trace, wherein the first voltage indicates that a first reagent comprising a first concentration of a detectable compound has been added to the calibration well; measuring a second electrical signal of the third calibration trace over a first calibration time period, wherein the third calibration trace is associated with the first reagent comprising the first concentration of the detectable compound; calibrating the reader to the first reagent on the first test strip based on the second electrical signal; detecting an addition of a first test sample in the first sample well based on measuring a third electrical signal on the second test trace, wherein the first test sample comprises the first reagent, and wherein an incubation period elapses before the first test sample is added to the first sample well, and a first biological sample is added into the first reagent during the incubation period and the first biological sample comprises an enzyme, wherein the enzyme catalyzes a reaction of the precursor compound to produce a second concentration of the detectable compound; measuring a fourth electrical signal of the third test trace over a first test time period; calculating the second concentration of the detectable compound in the first test sample; and reporting a diagnosis state based on the second concentration of the detectable compound.

In one embodiment, the electrical signal is voltage.

In one embodiment, the detectable compound is either glucose or paracetamol.

In one embodiment, the detectable compound is paracetamol.

In one embodiment, the second concentration of the detectable compound is either 1 mM or below 1 mM.

In one embodiment, the second concentration of the detectable compound is a concentration selected from the group consisting of 0.9 mM, 0.8 mM, 0.7 mM, 0.6 mM, 0.5 mM, 0.4 mM, 0.5 mM, 0.4 mM, 0.3 mM, 0.2 Mm, 0.1 mM and 0.05 mM.

### BRIEF DESCRIPTION OF THE FIGURES

FIG. 1 is a block diagram of a portable microbial load detection system according to an example of the present disclosure.

FIG. 2A is block diagram of a test strip in a portable microbial load detection system according to an example of the present disclosure.

FIG. 2B-D are time-lapse block diagrams of a test strip in a portable microbial load detection system during a microbial load test according to an example of the present disclosure.

FIG. 3 is a flowchart illustrating reader procedures in an example of portable microbial load detection according to an example of the present disclosure.

FIG. 4 is a flowchart illustrating operator procedures during portable microbial load detection according to an example of the present disclosure.

FIG. 5 is a flowchart illustrating an example of portable microbial load detection according to an example of the present disclosure.

FIG. 6 is a flow diagram illustrating an example system employing portable microbial load detection according to an example of the present disclosure.

FIGS. 7A-7D is a set of graphs illustrating the as-measured electrical signals (e.g., nanoamps) of the present device relating different concentrations of a detectable compound (e.g., paracetomol; mMol) according to an example of the present disclosure.

FIG. 8A is a graph illustrating comparative results between a standard curve of paracetamol using both a prototype meter according to an example of the present disclosure and a commercially available glucose meter. The standard curve in the range (0-5 mM): The prototype meter according to an example of the present disclosure can detect in this entire range (0-5 mM), however the commercially available glucose meter can detect the presence of the Paracetamol only in 1-5 mM range.

FIG. 8B is a graph illustrating another set of comparative results between a standard curve of paracetamol using both a prototype meter according to an example of the present disclosure and a commercially available glucose meter. The standard curve in the range (0-1 mM): The prototype meter according to an example of the present disclosure can detect in this range (0-1 mM) of the paracetamol and show a linear co-relation, however the commercially available glucose meter can only detect 1 mM or more of the paracetamol, and does not show any meaningful reading for any of the concentration below 1 mM for paracetamol (0-0.9 mM range).

### DETAILED DESCRIPTION OF EXAMPLE EMBODIMENTS

In typical medical, veterinary, or botanical diagnostic procedures, confirmation of infection by a particular microbe may result in significantly improved treatment options for a patient, whether human, animal, or plant. Typically, computing the actual microbial concentration in the patient may allow for even better dosing of medication to combat the infection. However, typically, such tests may be expensive and may be performed by large laboratory based equipment that may be unavailable at a doctor's, veterinarian's, or botanist's office or in the field anywhere where an infectious outbreak may occur. Therefore, in many typical scenarios, a test is only ordered after a patient does not respond to generic treatment options, which presents an inefficient solution for both doctor and patient, requiring extra doctor visits and disruptions to the patient's schedule. Typically, microbial load testing is standard procedure only where such testing is a vital part of a given treatment regime for a chronic condition. For example, monitoring of the concentration of virus or viral load in human immunodeficiency virus (“HIV”) positive patients is standard procedure, while testing for influenza virus may not be conducted until a follow-up visit after a patient with flu-like symptoms does not initially recover.

In a typical example, microbial load, especially viral load may be measured through some form of nucleic acid testing, where Deoxyribose Nucleic Acid (“DNA”) or Ribonucleic Acid (“RNA”) is extracted from a biological sample (e.g., blood, saliva, mucus, biopsy, etc.) and then a measurement of the DNA/RNA concentration of the microbe is converted into a measurement of microbial concentration. For example, known sections of microbial DNA/RNA may be amplified in a controlled manner (e.g., polymerase chain reaction) and then probes (e.g., radioactive or optical probes) may be attached to the amplified nucleic acid to be measured. In another example, a probe may be attached to unamplified DNA/RNA samples, and after removal of unbound probes, the remaining probes may instead be amplified to measureable levels. In other examples, large amounts of secondary probes may be flooded in that bind to primary probes binding DNA/RNA to reach measurable concentrations. In all of these examples, large, dedicated laboratory equipment (e.g., thermocyclers, centrifuges, nucleic acid extraction equipment, etc.) are required, along with significant hands-on preparation time in order to measure microbial load. In addition, typical nucleic acid based microbial load testing may require specialized training in techniques and equipment outside the skill set of a typical nurse or clinician, adding another potential impediment to broad deployment.

It is possible through various biological and/or chemical assays to cause infectious microbes to generate detectable byproducts through catalyzed reactions. In an example, a microbe in the presence of certain reagents and certain incubation conditions may generate glucose, which may be detectable through a glucose meter. In the example, the concentration of glucose measured may then be converted into a corresponding microbial load measurement. However, while glucose measurements may be useful in a sterile laboratory environment, glucose is typically present in vastly larger quantities inside of living organisms than the amounts typically generated by such microbial assays, and therefore isolating for microbial related glucose readings in a clinical environment may be impractical.

In addition, typical glucose meters (e.g., for diabetic patients) lack the sensitivity to measure such low quantities of glucose (e.g., below 1 mM or even below 0.1 mM). In an example, an alternative product may be generated by the biological and/or chemical assays, for example, a small pharmaceutical compound such as paracetamol or acetaminophen. However, portable, clinical testing of the concentration (e.g., a low concentration such as below 1 mM or even below 0.1 mM) of such small molecule compounds as detectable compounds may be unavailable as well, due to the equipment typically required. For example, the levels of signal generated from such reactions and low concentrations (e.g., a low concentration such as below 1 mM or even below 0.1 mM of paracetamol) may typically be below the threshold for background noise in a typical portable detector, such as a commercially available glucose meter for diabetic patients. For example, as demonstrated in the Example 2, a commercial glucose meter (e.g., One Touch Ultra 2 meter) can be used to test for paracetamol rather than glucose. A commercial glucose meter (e.g., One Touch Ultra 2 meter) is known to measure 1 mM or greater than 1 mM glucose. A commercial glucose meter (e.g., One Touch Ultra 2 meter) cannot measure with sufficient reliability or precision a concentration below 1 mM of glucose. As shown in the Example 2, the commercially available glucose meter (e.g., One Touch Ultra 2 meter) can only detect 1 mM or greater than 1 mM of the paracetamol, and does not show any meaningful reading for any of the concentration below 1 mM for paracetamol (0-0.9 mM range).

The present disclosure aims to address the detection and monitoring of microbial load in clinical settings, accurately and efficiently. For example, the present system or the related method of using such a system is capable of detecting a low concentration (e.g., below 1 mM or even below 0.1 mM) of a small molecule compound as a detectable compound. The examples of the detectable compound may include glucose, paracetamol or any small organic molecule indicative of or correlative with an amount of a virus (e.g., influenza virus) or other microbial in a sample. In one embodiment, the small molecule compound is glucose. In one embodiment, the present system or the related method of using such a system is capable of detecting a low concentration such as below 1 mM of glucose as the detectable compound. In another embodiment, the small molecule compound is paracetamol. In one embodiment, the present system or the related method of using such a system is capable of detecting a low concentration such as below 1 mM of paracetamol as the detectable compound.

For example, a high-sensitivity reader device of a form factor between the size of a smart phone and a laptop of the present disclosure may be combined with specialized test strips and reagent dispensers to allow for minimally or non-invasive microbial load tests to be conducted during a typical doctor's visit. In an example, a test strip may be individually calibrated to a background noise level associated with a particular sample of catalyzing a reagent comprising a precursor compound to form the detectable compound and/or a background level of the detectable compound in a biological sample from a patient. Precise calibration of the system of the present disclosure allows for detection of signal levels (corresponding to concentrations of the detectable compound such as paracetamol) that may be similar in magnitude to background noise in typical detector devices. The reader and/or the test strip of the system of the present disclosure may then provide stimuli to drive forward the reaction to generate the detectable compound, for example, through enzymes and/or a drive signal such as electricity or light.

In one embodiment, the precursor compound may be included anywhere in the present system. In one embodiment, a related enzyme (which may catalyze the precursor compound to form the detectable compound) may be included anywhere in the present system. The precursor compound and the related enzyme may only co-exist in the same location of the present system right before a measurement so that the precursor compound can be catalyzed to produce the detectable compound.

For example, the precursor compound may be included anywhere in the present system, e.g., in any strip (test strip, reagent strip or any other strip) so that a reaction for producing paracetamol may proceed when the strip is in contact with a sample comprising the related enzyme. The related enzyme may be later added into the system or a sample. Alternatively, the related enzyme may be included anywhere in the present system. For example, the related enzyme may be included in any strip (test strip, reagent strip or any other strip) or any other part of the present system. The precursor compound may be later added into the system or a sample so that a reaction for producing paracetamol may proceed when the related enzyme is in contact with the precursor compound.

In one embodiment, the precursor compound of the present system (e.g., on the strip and/or in the fluid dropped on the strip/in the well) may not need any external stimuli (e.g., electrical signal or light) to form the detectable compound. In another embodiment, an external stimuli (e.g., electrical signal, light, Laser, ultrasonic, microwave, or any other driving force) may be used to drive forward the reaction to form the detectable compound for measurement. For example, an electrical signal may be needed for the measurement of a detectable compound such as paracetamol.

In one embodiment, the reaction to form the detectable compound might not need any external stimuli to occur. For example, an enzyme catalytic reaction may not need any external stimuli to occur. In another embodiment, although the reaction to form the detectable compound may occur at a slower rate even without any electric/optical stimuli, the reaction may be sped up in the presence of the external stimuli (e.g., electrical signal, light, Laser, ultrasonic, microwave, or any other driving force). An external stimuli (e.g., electrical signal, light, Laser, ultrasonic, microwave, or any other driving force) driving a chemical reaction was well known in the art. For example, light or Laser is known to drive a reaction of a photo-sensitive compound. A microwave driven reaction is also well known in the art.

An external stimuli such as electrical signal driving a reaction is also well known in the art. For example, an external stimuli such as electrical currents flowing through the sample, optical signals or light can drive a reaction.

In one embodiment, when the detectable compound is paracetamol, the precursor compound may be 4,7-methoxy sialic acid-paracetamol (SP(OMe)₂). The detailed synthesis of (SP(OMe)₂) was described in Anal. Chem. 2018, 90, 19, 11589-11598. In one embodiment, SP(OMe)₂ may be cleaved by influenza viruses or its enzyme, Neuraminidase (NA) to release paracetamol.

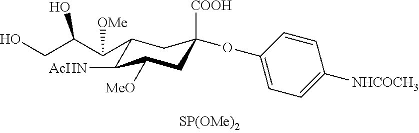

In one embodiment, other precursor compound can be used in the present invention. For example, glucose-paracetamol (GP) was also synthesized in Anal. Chem. 2018, 90, 19, 11589-11598. GP can also be used as a precursor compound of the present invention. For example, GP can be cleaved by an enzyme (beta-galactosidase) produced by E. coli to produce both paracetamol and glucose.

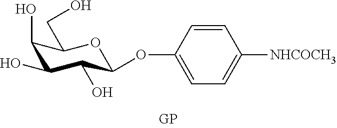

In one example, measurements of the present system of the generated detectable compound such as paracetamol may be template matched to known control measurements and a microbial load on the basis of the concentration of the detectable compound such as paracetamol may then be calculated. Calibration on a particular test strip and a particular aliquot of a reagent (e.g., a reagent comprising a known concentration of the detectable compound such as paracetamol) allows for background noise that may affect results to be filtered out. As a result, a doctor may provide better diagnosis and better treatment options for the type of infection affecting the patient. With a highly portable form factor and power requirements low enough to be supplied by batteries or solar power, microbial load detection of the present system may be brought as needed to a patient, anywhere in the world without worrying about transportation of samples to a lab. A test that may typically take a three day turn around and require expedited temperature controlled shipping may be performed by using the present system in 15 minutes on a reader that a doctor carries in a backpack on house calls. By using the presently disclosed system and methods, doctors may be able to improve patient outcomes by reducing the spread of disease, better treating infectious disease, and potentially saving lives.

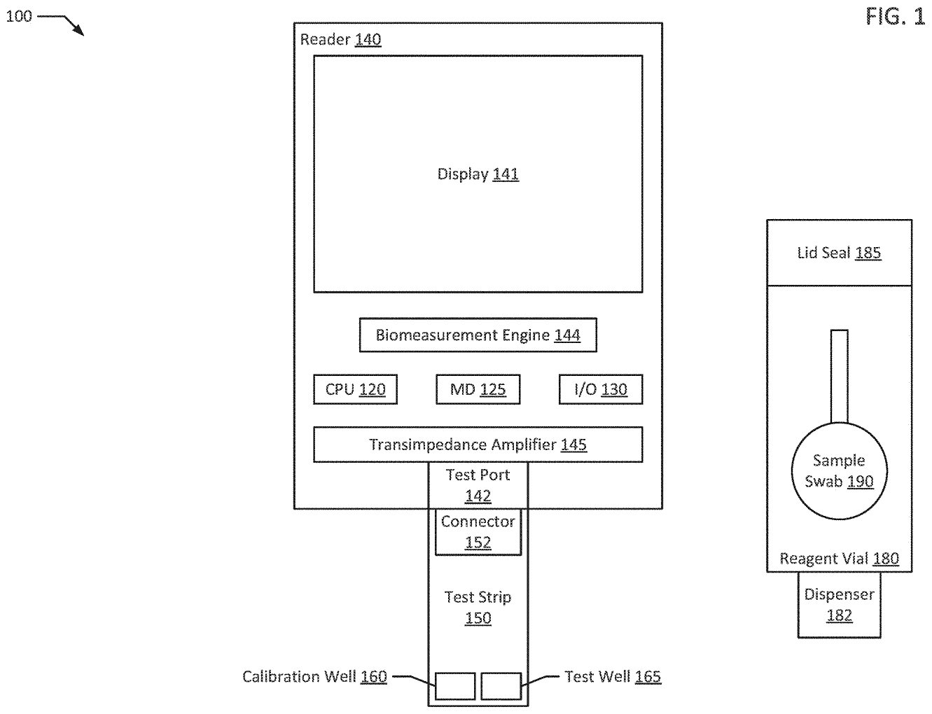

FIG. 1 is a block diagram of a portable microbial load detection system according to an example of the present disclosure. The system 100 may include a reader 140, which may be include one or more physical processors (e.g., CPU 120) communicatively coupled to memory devices (e.g., MD 125) and input/output devices (e.g., I/O 130). As used herein, physical processor or processors (Central Processing Units “CPUs”) 120 refer to devices capable of executing instructions encoding arithmetic, logical, and/or I/O operations. In one illustrative example, a processor may follow Von Neumann architectural model and may include an arithmetic logic unit (ALU), a control unit, and a plurality of registers. In an example, a processor may be a single core processor which is typically capable of executing one instruction at a time (or process a single pipeline of instructions), or a multi-core processor which may simultaneously execute multiple instructions. In another example, a processor may be implemented as a single integrated circuit, two or more integrated circuits, or may be a component of a multi-chip module (e.g., in which individual microprocessor dies are included in a single integrated circuit package and hence share a single socket). A processor may also be referred to as a central processing unit (CPU).

As discussed herein, a memory device 125 refers to a volatile or non-volatile memory device, such as RAM, ROM, EEPROM, or any other device capable of storing data. As discussed herein, I/O device 130 refers to a device capable of providing an interface between one or more processor pins and an external device, the operation of which is based on the processor inputting and/or outputting binary data. CPU 120 may be interconnected using a variety of techniques, ranging from a point-to-point processor interconnect, to a system area network, such as an Ethernet-based network. Local connections within reader 140, including the connections between a CPU 120 and memory device 125 and between CPU 120 and an I/O device 130 may be provided by one or more local buses of suitable architecture, for example, peripheral component interconnect (PCI). In an example, display 141 may be a visual display output interface (e.g., liquid crystal display (“LCD”)). In an example, display 141 may be an input interface (e.g., touchscreen), connected to I/O device 130. In another example, display 141 may both be a visual output LCD and an input touch screen. In an example, a separate keyboard may be provided as an input device.

In an example, biomeasurement engine 144 may be software or hardware configured to take measurement values from transimpedance amplifier 145 and to compute a compound concentration based on electrical signal readings such as voltage readings generated by transimpedance amplifier 145. In an example, biomeasurement engine 144 may be implemented via any form of executable code (e.g., executable file, script, application, service, daemon). In an example, the biomeasurement engine 144 may be implemented as an application-specific integrated circuit (“ASIC”). In an example, biomeasurement engine 144 may be further configured to perform template matching to correlate detectable compound concentrations with medical diagnosis. In an example, transimpedance amplifier 145 may be a hardware device that converts current to voltage for reader 140. In an example, transimpedance amplifier 145 may be implemented with an operational amplifier. In some examples, reader 140 may be equipped with alternative components to transimpedance amplifier 145 for receiving readings from test strip 150. In an example, any suitable component of sufficient sensitivity for measuring current or voltage may be substituted for transimpedance amplifier 145 (e.g., picoammeter, ammeter, voltmeter, oscilloscope, current transformer, isolation amplifier, etc.).

In another example, reader 140 may measure a different input other than electrical current and/or amperage. For example, if test strip 150 is configured for a light based drive signal rather than an electrical drive signal, transimpedance amplifier 145 may be replaced with a photosensor if a photosensitive probe is used. If a radioactive probe is used, an appropriate radiation detector may also be substituted. In an example, the drive signal may be of a different type of energy than the detection device. For example, a light catalyzed reaction may be implemented with a laser for a drive signal but may still measure electrical charge, (e.g., via transimpedance amplifier 145) for the output of the reaction. In an example, multiple types of drive signal may be combined to create specific conditions for a given reaction, and the one or more drive signals may be modulated and/or scaled during the course of a test to progress the reaction. For example, a reaction may perform best with heating and cooling cycles (e.g., polymerase chain reaction).

Another reaction may perform best with both heat and light as drive signals. In an example, transimpedance amplifier 145 receives input from test port 142, which may be any form of suitable connection port into which test strip 150 may be connected via connector 152. In an example, test port 142 may have separate electrical traces to different channels in transimpedance amplifier 145, each associated with separate electrical traces in test strip 150 exposed by connector 152. In the example with an optical or photosensitive test strip, test port 142 and its mating piece, connector 152 may be any form of suitable optical interface.

In an example, test strip 150 may be a disposable, biologically sealed, and sterile test strip with electrical traces leading to at least two wells in which samples may be placed (e.g., calibration well 160 and test well 165). In an example, calibration well 160 and test well 165 may be biologically isolated from each other, for example, to avoid cross contamination. In an example, reagent vial 180 may be a single use reagent container containing a suitable reagent such as a precursor compound which, when catalyzed by an enzyme in a biological sample on sample swab 190, may generate a detectable small organic molecule such as paracetamol as the detectable compound.

In another embodiment, an amount of the detectable small organic molecule such as paracetamol as the detectable compound may pre-exist in the biological sample on sample swab 190. The precursor compound of reagent vial 180, when catalyzed by an enzyme in a biological sample on sample swab 190, may generate some additional paracetamol as the detectable compound. The present system is capable of detecting the amount of the pre-existing detectable compound and/or the amount of the additional detectable compound. In one embodiment, the present system or the related method of using such a system is capable of detecting a low concentration such as below 1 mM of paracetamol as the detectable compound.

In an example, the reagent in reagent vial 180 may be in liquid form. In another example, the reagent may be reconsitutable with a suitable solvent (e.g., water, alcohol, etc.) or with a liquid biological sample (e.g., blood, mucus, tears). In an example, a solid biological sample (e.g., skin cells, biopsy sample, etc.) may be immersed in reagent vial 180.

In an example, reagent vial 180 may include a lid seal 185, which may have a one-way entrance through which sample swab 190 may be inserted. In an example, dispenser 182 may be configured to mate with calibration well 160 and/or test well 165. In an example, test strip 150 may have a separate injection port that may mate with dispenser 182 for channeling reagent and/or sample to calibration well 160 and test well 165. In an example, an automated fluidics system may channel reagent and/or sample to calibration well 160 and/or test well 165. In an example, incubation of the biological sample may occur in reagent vial 180. In another example, incubation of the biological sample may occur in a separate incubation chamber, for example, on test strip 150 or reader 140. In an example, dispenser 182 may be configured to accurately dose injection amounts into calibration well 160 and/or test well 165. For example, a proper amount of reagent and/or sample may be dispensed with a twist of reagent vial 180, or a press of a button, etc.

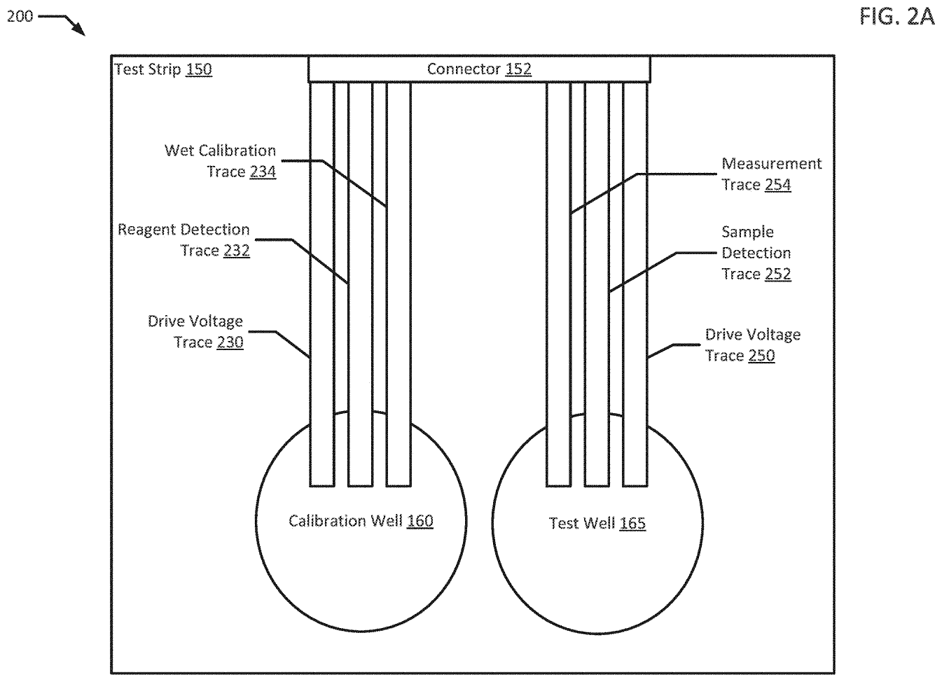

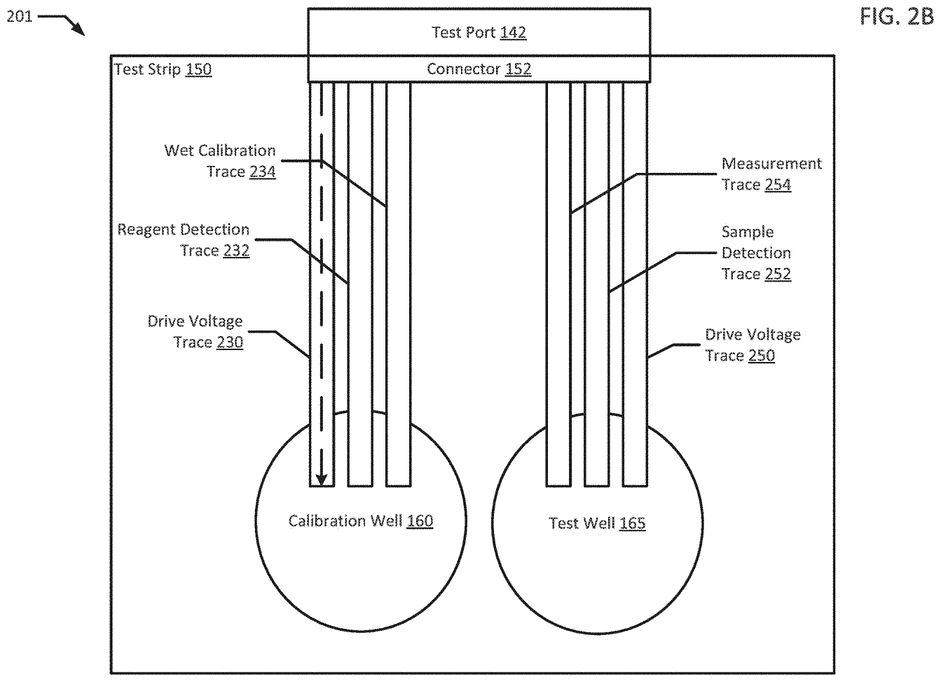

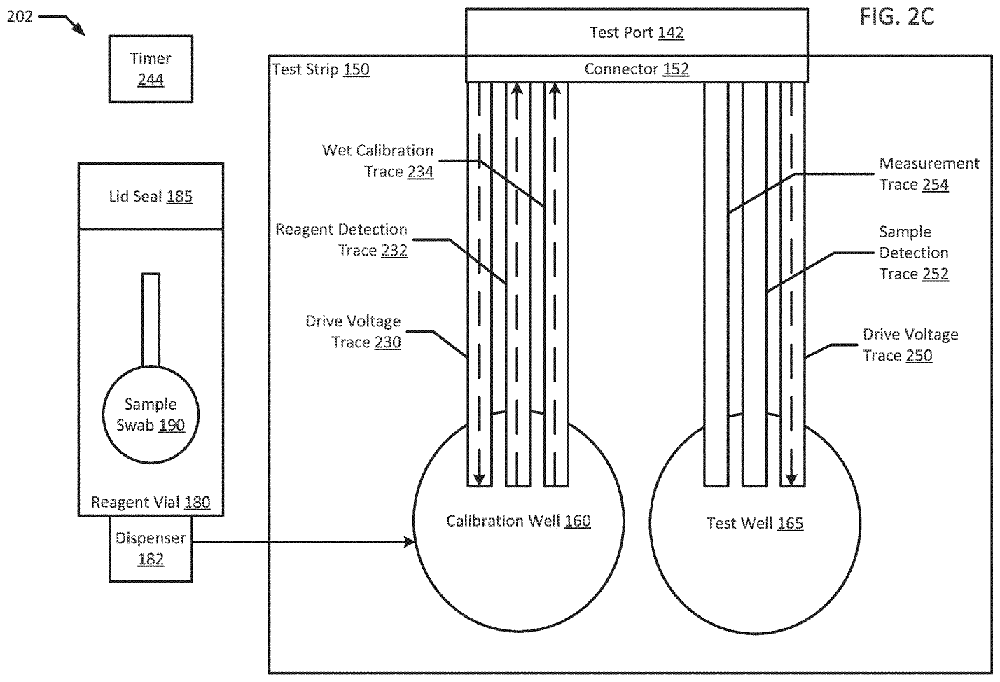

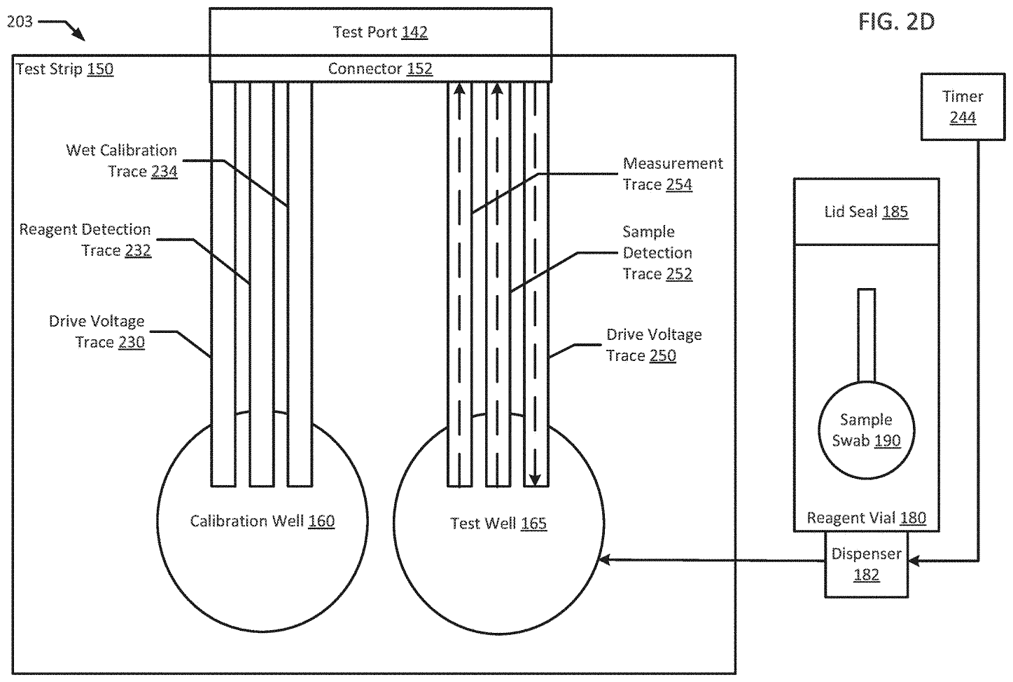

FIG. 2A is a block diagram of a test strip in a portable microbial load detection system according to an example of the present disclosure. Exemplary system 200 may be an enlarged illustration of test strip 150 from system 100. In an example, at least three electrical traces lead from connector 152 to each of calibration well 160 and test well 165. In some examples, a test strip may have multiple test wells each with their own respective set of electrical traces. In some examples, each well may have additional traces for additional measurements beyond three traces shown in system 200. In an example, calibration well 160 is connected to drive voltage trace 230, reagent detection trace 232, and wet calibration trace 234. In an example, test well 165 is connected to drive voltage trace 250, sample detection trace 252, and measurement trace 254. In an example, each trace (e.g., drive voltage trace 230, reagent detection trace 232, wet calibration trace 234, drive voltage trace 250, sample detection trace 252, and measurement trace 254) may be constructed of any suitable conductive material.

In an example, each of drive voltage trace 230, reagent detection trace 232, wet calibration trace 234, drive voltage trace 250, sample detection trace 252, and measurement trace 254 may be insulated from each other trace. In an example, each of drive voltage trace 230, reagent detection trace 232, wet calibration trace 234, drive voltage trace 250, sample detection trace 252, and measurement trace 254 may be wired to a separate connection device (e.g., pin) in connector 152. In an example, each of drive voltage trace 230, reagent detection trace 232, wet calibration trace 234, drive voltage trace 250, sample detection trace 252, and measurement trace 254 may correspond to a separate channel in transimpedance amplifier 145. In other examples, signals from multiple traces may be multiplexed together via any suitable method, for example, where there are more traces in test strip 150 than there are channels in transimpedance amplifier 145.

FIG. 2B-D are time-lapse block diagrams of a test strip in a portable microbial load detection system during a viral load test according to an example of the present disclosure. System 201 as illustrated in FIG. 2B depicts the test strip 150 from system 200 after test strip 150 is plugged into test port 142 of reader 140. In an example, reader 140 detects that test strip 150 has been plugged in. For example, reader 140 may detect a transient electrical pulse (e.g., from static electricity) indicating that test strip 150 has been plugged into test port 142. In an example, the transient electrical pulse may be detected on any of drive voltage trace 230, reagent detection trace 232, wet calibration trace 234, drive voltage trace 250, sample detection trace 252, and measurement trace 254. In an example, in response to detecting that test strip 150 is plugged in, reader 140 begins sending a drive voltage through drive voltage trace 230. In some examples, drive voltage trace 250 may begin receiving the drive voltage as well. In an example, drive voltage traces 230 and 250 may be connected to a same trace. In another example, where the drive signal is not a drive voltage, the alternative drive signal may be applied (e.g., a laser or other light source).

FIG. 2C and system 202 illustrate system 201 as a reagent is added to calibration well 160. In an example, when a first drop of the reagent is dispensed from dispenser 182 to calibration well 160, the drive voltage from drive voltage trace 230 shorts to reagent detection trace 232 and/or wet calibration trace 234. In the example, upon detection of voltage from reagent detection trace 232 by transimpedance amplifier 145, or alternatively upon a user confirmation that calibration reagent has been added to calibration well 160, a timer 244 is started for when a sample should be added to test well 165. In addition, biomeasurement engine 144 may begin recording a series of voltage or current readings from transimpedance amplifier 145 and wet calibration trace 234 over a calibration window. In the example, the voltage or current from wet calibration trace 234 may then be factored into any later readings from test well 165 and measurement trace 254 to calibrate for background noise generated by, for example, manufacturing differences between tests strips or between reagent batches.

In an example, if the measurements from wet calibration trace 234 are too far off from a set baseline, test strip 150 may be rejected as defective. In an example, sample swab 190 may be inserted into reagent vial 180 before the reagent is added to calibration well 160. For example, in a slow acting reaction, introducing biological sample into the calibration well may be undetectable during the calibration time period, but may allow the wet calibration process to factor in any variations between different sample swabs. In another example, reagent may be dispensed through dispenser 182 to calibration well 160 before sample swab 190 is inserted into reagent vial 180 to ensure that only background noise from the reagent and test strip are factored in, and that any biological sample cannot affect the calibration process. For example, if the observed reaction is fast, early insertion of the biological sample may significantly skew the calibration data. In an example, drive voltage may be applied to drive voltage trace 250 after calibration is complete, for example, to detect early sample injection in test well 165. In another example, drive voltage 250 may be activated as timer 244 elapses.

FIG. 2D and system 203 illustrate system 201 where timer 244 has sounded. In an example, timer 244 alerts an operator to deposit incubated reagent/biological sample mix from reagent vial 180 to test well 165, for example, after 15 minutes. In an example, dispensing reagent/sample mix into test well 165 shorts the drive voltage from drive trace 250 to sample detection trace 252 and measurement trace 254. In an example, upon detecting voltage from sample detection trace 252, biomeasurement engine 144 begins recording voltage or amperage readings from measurement trace 254 over a measurement window. In an example, any time after test strip 150 is calibrated based on wet calibration trace 234, drive voltage trace 230 may be turned off, for example, to conserve battery power. In the example, the recorded readings from measurement trace 254 may be compared with existing known values after adjustments based on the wet calibration process in system 202. In an example, biomeasurement engine 144 computes a microbial load value for the sample on sample swab 190 and reports a diagnosis on display 141.

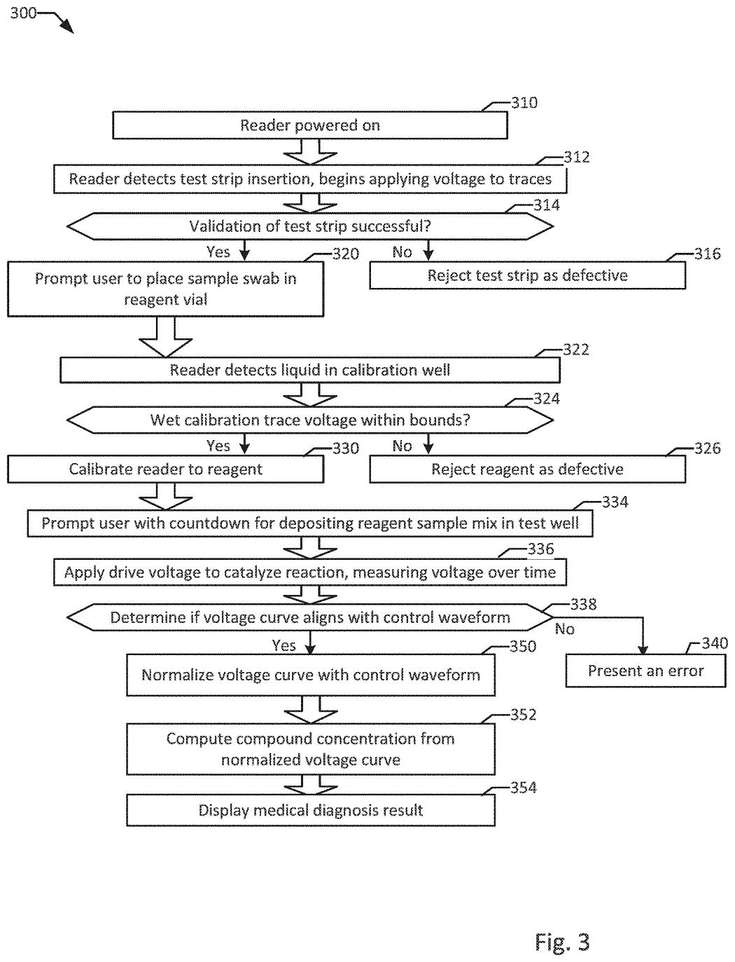

FIG. 3 is a flowchart illustrating reader procedures in an example of portable microbial load detection according to an example of the present disclosure. Although the example method 300 is described with reference to the flowchart illustrated in FIG. 3, it will be appreciated that many other methods of performing the acts associated with the method 300 may be used. For example, the order of some of the blocks may be changed, certain blocks may be combined with other blocks, and some of the blocks described are optional. The method 300 may be performed by processing logic that may comprise hardware (circuitry, dedicated logic, etc.), software, or a combination of both. In an example, the method is performed by reader 140.

In an example, reader 140 is powered on (block 310). In the example, reader 140 detects the insertion of test strip 150 into test port 142, for example, due to a transient electrical pulse (block 312). In an example, reader 140 feeds in a drive voltage on drive voltage trace 230 and validates whether the test strip 150 is genuine and free of defects (block 314). For example, the drive voltage may be measured to ensure that there is no short between any of drive voltage trace 230, reagent detection trace 232, wet calibration trace 234, drive voltage trace 250, sample detection trace 252, and measurement trace 254. In an example, a wiring short may be distinguishable from a short due to liquid in a well because a wiring short may be between traces that would not short to each other from a sample. In an example, if drive voltage trace 230 shorts to, for example, wet calibration trace 234 but not to reagent detection trace 232, the test strip 150 is rejected as defective (block 316). Additional security measures may be present, such as an RFID for authentication between reader 140 and test strip 150. In an example, upon successful validation of test strip 150, a user is prompted to place sample swab 190 into reagent vial 180 (block 320). The user may additionally be prompted to mix per instructions on display 141 and/or to dispense reagent into calibration well 160. In an example, reader 140 may detect liquid in calibration well 160 (block 322). In the example, reader 140 may detect whether a voltage of wet calibration trace 234, now shorted to drive voltage trace 230, is within bounds (block 324). In an example, if water is spilled into calibration well 160, the conductive properties may be different from the reagent/sample mixture in reagent vial 180. In an example, reagent vial 180 may have been previously contaminated with microbes and the detectable compound may already be present in high concentration leading to unrealistic readings. In the example, reagent vial 180 may be rejected as defective (block 326). In an example, biomeasurement engine 144 may determine that calibration trace 234 is measuring a voltage within calibration limits, and may calibrate reader 140 to the reagent/test strip combination (block 330).

In an example, a user is prompted via a count down to deposit reagent/sample mix into test well 265, for example, via timer 244 on display 141 (block 334). Reader 144 may apply drive voltage to drive voltage trace 250 any time after test strip 150 is inserted into test port 142. In an example, reader 140 applies drive voltage to catalyze a reaction between the biological sample and the reagent, measuring the voltage on measurement trace 254 over time (block 336). In an example, test well 165 and/or any of the traces connected to test well 165 (e.g., drive voltage trace 250, sample detection trace 252, and/or measurement trace 254) may be coated with a catalyst such as an enzyme that aids the reaction between the biological sample and the reagent in test well 165. In an example, the voltage readings from measurement trace 254 are plotted over time, and a resulting voltage curve is compared to a control waveform for an infectious disease for which reader 140 is configured to detect (block 338). In the example, if the voltage curve does not match the control waveform(s), reader 140 presents an error (block 340). In an example, the voltage curve is normalized with the control waveform(s), for example, after factoring in the reader calibration (block 350). In an example, the detectable compound concentration is computed from the normalized voltage curve (block 352). In an example, a medical diagnosis result is displayed on display 141 (block 354). In an example, the actual measured microbial load concentration and the detectable compound concentration may be obfuscated, with only a computed diagnosis displayed. For example, possible outputs may be infected, not infected, and inconclusive. In some examples, where a certain threshold of microbial load may be a threshold for differential treatment, such thresholding may additionally be displayed.

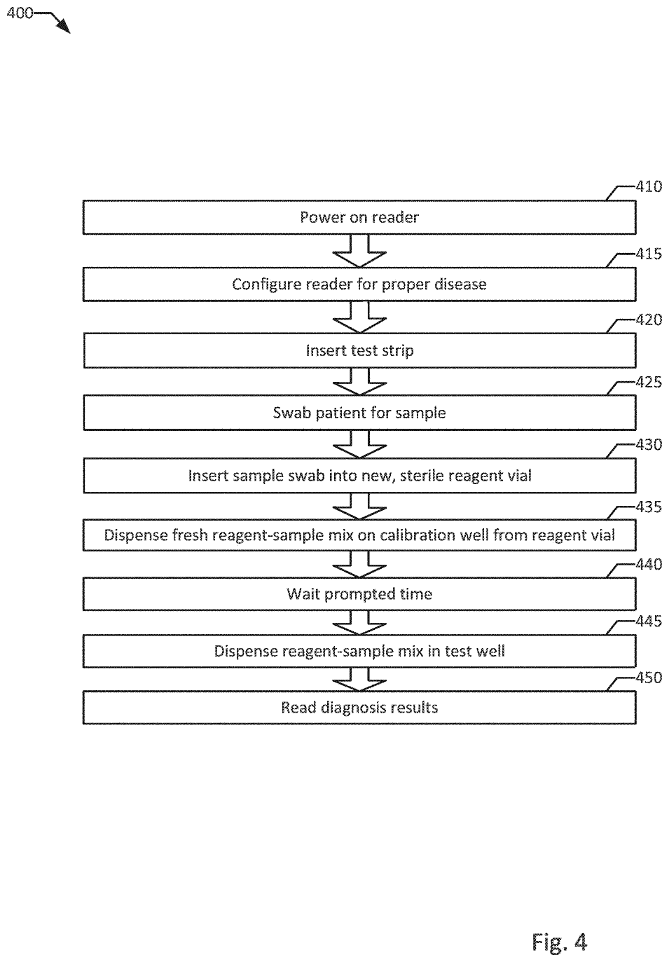

FIG. 4 is a flowchart illustrating operator procedures during portable microbial load detection of the present system according to an example of the present disclosure. Although the example method 400 is described with reference to the flowchart illustrated in FIG. 4, it will be appreciated that many other methods of performing the acts associated with the method 400 may be used. For example, the order of some of the blocks may be changed, certain blocks may be combined with other blocks, and some of the blocks described are optional. The method 400 may be performed by processing logic that may comprise hardware (circuitry, dedicated logic, etc.), software, or a combination of both. In an example, the method is performed by an operator operating reader 140.

In example system 400, an operator (e.g., medical technician, nurse, doctor, etc.) may power on reader 140 (block 410). The operator may then configure reader 140 for the proper disease being tested (e.g., in relation to reagent vial 180 and/or test strip 150) (block 415). For example, the reader 140 may be configured to an influenza viral load test, or an HIV viral load test, a Staphylococcus aureus bacterial load test, an E. coli bacterial load test, a Candida fungal load test, etc. In an example, the operator inserts test strip 150 into reader 140 (block 420). In an example, the operator swabs a patient for a sample (e.g., in a nostril, on the inside of the cheek, or another mucus membrane) (block 425). In an example, the sample swab 190 is inserted into a new, sterile reagent vial 180 (block 430). In an example, the fresh reagent-sample mix is dispensed in calibration well 160 from reagent vial 180 (block 435). In the example, the operator then waits the prompted amount of time (block 440). The operator then dispenses the reagent-sample mix into test well 165 (block 445). In an example, the operator finally reads the medical diagnosis from display 141 (block 450).

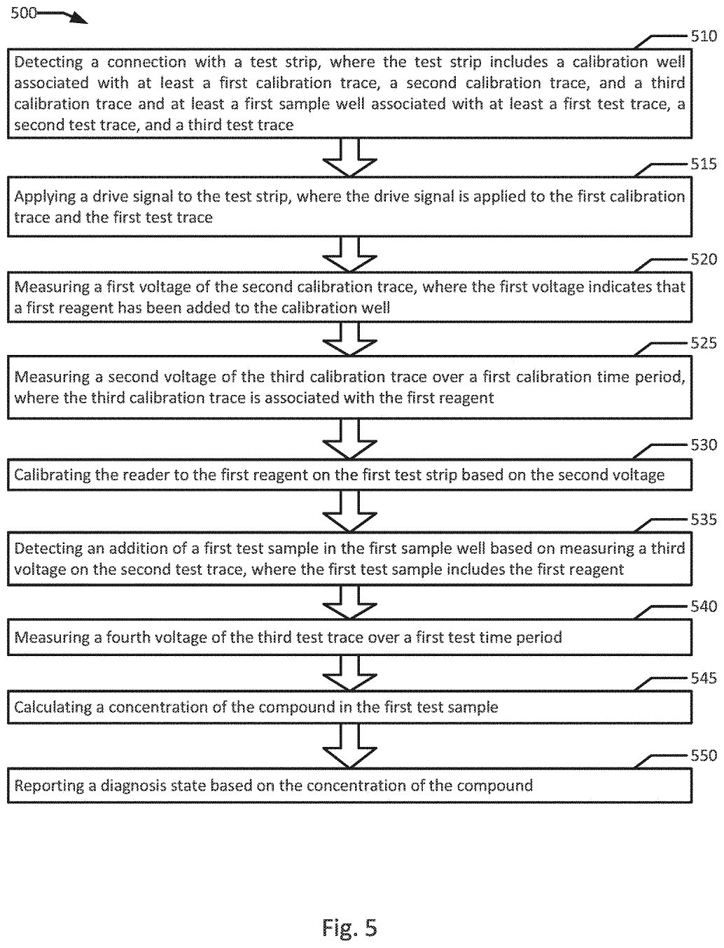

FIG. 5 is a flowchart illustrating an example of portable microbial load detection of the present system according to an example of the present disclosure. Although the example method 500 is described with reference to the flowchart illustrated in FIG. 5, it will be appreciated that many other methods of performing the acts associated with the method 500 may be used. For example, the order of some of the blocks may be changed, certain blocks may be combined with other blocks, and some of the blocks described are optional. The method 500 may be performed by processing logic that may comprise hardware (circuitry, dedicated logic, etc.), software, or a combination of both. In an example, the method is performed by reader 140.

The example method 500 may begin with detecting a connection with a test strip, where the test strip includes a calibration well associated with at least a first calibration trace, a second calibration trace, and a third calibration trace and at least a first sample well associated with at least a first test trace, a second test trace, and a third test trace (block 510). In an example, reader 140 detects a connection through test port 142 and connector 152 with test strip 150, for example, due to measuring a transient electrical pulse from static electricity. As a result, reader 140 may begin applying a drive signal to the test strip 150, where the drive signal is applied to the first calibration trace (e.g., drive voltage trace 230) and the first test trace (e.g., drive voltage trace 250) (block 515). In an example, the drive signal may be applied sequentially to drive voltage trace 230 and then to drive voltage trace 250 as needed rather than concurrently, for example, to conserve power. In an example, drive signal may be shut off to calibration well 160 any time after calibration is complete, and drive signal may be started for test well 165 any time before testing is set to occur, for example, based on timer 244.

In some examples, multiple drive signals may be implemented and may be configured to optimally catalyze a reaction. For example, certain reactions may be catalyzed by light, electricity, heat, etc. or a combination of factors. In various examples, the drive signal may be an electrical pulse, an optical pulse, or any other motive force and/or catalytic force applied to test strip 150. In an example, multiple drive signals may be turned on sequentially or concurrently in a combination most suited to the reaction being tested. In an example, reader 140 measures a first voltage of the second calibration trace (e.g., reagent detection trace 232), where the first voltage indicates that a first reagent has been added to the calibration well 160 (block 520). In an example, adding a dissolved reagent to calibration well 160 shorts drive voltage trace 230 to reagent detection trace 232. In an example, reagent in reagent vial 180 may be in any suitable form, including liquid, gel, solid, lyophilized, etc., and may be reactive or may serve a labeling purpose (e.g., colormetric dye). In an example, reagent vial 180 may contain necessary ingredients in the form of organic precursor molecules (e.g., precursor compounds for producing the detectable compounds) for a reaction catalyzed (e.g., by an enzyme present) in test well 165. In an example, reagent vial 180 may contain photo or radioactive probes detectable via optical or radiation sensors rather than electrical sensors.

In an example, the drive voltage may be of any suitable magnitude. In a typical example, a drive voltage producing 10 milliamps to 1000 milliamps of current in drive voltage trace 230 and/or drive voltage trace 250 may be preferential depending on the underlying assay being measured. Certain reactions may perform optimally with lower or higher drive voltages. In an example, a drive voltage producing around 200 milliamps of current on drive voltage traces 230 and 250 may be effective for the quantification of influenza viral load with a reaction generating paracetamol as the measured small molecule organic compound. In an example, an operational amplifier such as transimpedance amplifier 145 may be configured to convert current to voltage for the measurement of voltages on the various traces in test strip 150 (e.g., drive voltage trace 230, reagent detection trace 232, wet calibration trace 234, drive voltage trace 250, sample detection trace 252, and measurement trace 254). In an example, each of drive voltage trace 230, reagent detection trace 232, wet calibration trace 234, drive voltage trace 250, sample detection trace 252, and measurement trace 254 is connected to a separate channel in the transimpedance amplifier 145. In another example, at least two signals corresponding respectively to at least two of the drive voltage trace 230, reagent detection trace 232, wet calibration trace 234, drive voltage trace 250, sample detection trace 252, and measurement trace 254 are multiplexed together in the transimpedance amplifier 145. In an example, transimpedance amplifier 145 in a first configuration mode measures a first signal type associated with a first tested medical condition (e.g., influenza) associated with the first biological sample, and the transimpedance amplifier 145 in a second configuration mode measures a second signal type associated with a second tested medical condition (e.g., HIV, Ebola, tuberculosis, etc.) associated with a second biological sample.

In an example, reader 140 measures a second voltage of the third calibration trace (e.g., wet calibration trace 234) over a first calibration time period, where the third calibration trace is associated with the first reagent (block 525). In an example, the wet calibration trace 234 generates a baseline measurement factoring in the specific aliquot of reagent in reagent vial 180, the specific electrical peculiarities of test strip 150, and any background affecting factors from sample swab 190 and a biological sample (e.g., saliva) collected on sample swab 190 for a calibration reading, which may be a series of voltage or amperage readings over a calibration time period. In an example, reader 140 is calibrated to the first reagent (e.g., the reagent in reagent vial 180) on the first test strip (e.g., test strip 150) based on the second voltage (block 530). In an example, a second reading of calibration well 160 may be taken at a later, intermediate time as a validation reading against contamination. For example, for a test that takes 15 minutes, if a 5 minute reading is already high, at least one of test strip 150, reagent vial 180, and reader 140 is likely defective. In some examples, an initial incubation or settling period may be taken after a sample is added to sample vial 180 before the calibration well 160 is filled.

In an example, reader 140 detects an addition of a first test sample (e.g., a mixture of biological sample and reagent from reagent vial 180) in the first sample well (e.g., test well 165) based on measuring a third voltage on the second test trace (e.g., sample detection trace 252, which may be shorted to drive voltage trace 250 via the first test sample), where the first test sample includes the first reagent (block 535). In an example, an incubation period elapses before the first test sample is added to test well 165, and the biological sample is in contact with the first reagent during the incubation period. In an example, a non-liquid reagent may first be dissolved with an appropriate solvent. In an example, reader 140 measures a fourth voltage of the third test trace (e.g., measurement trace 254) over a first test time period (block 540). In an example, the reagent-sample mix in test well 165 shorts drive voltage trace 250 to measurement trace 254. In an example, a reaction between a biological sample (e.g., comprising a precursor compound) and the reagent (e.g., comprising an enzyme) in reagent vial 180 generates the detectable compound (e.g., paracetamol) being measured by biomeasurement engine 144.

In an example, the reaction is further catalyzed by a catalyst (e.g., an enzyme) in test well 165 and/or any of the traces on test strip 150. In an example, the catalyst (e.g., an enzyme) is also in calibration well 160 so that the catalyst may be calibrated for. For example, a sample reagent/enzyme combination may generate paracetamol as the detectable compound when in the presence of influenza and the reaction may be sped up by the application of an electrical drive signal. In an example, reagent/enzyme/current combinations may be customized for different microbes including viruses, bacterial, and fungi.

In an example, reader 140 calculates a concentration of the detectable compound (e.g., paracetamol) in the first test sample (block 545). In an example, the detectable compound is glucose. In another example, the detectable compound is paracetamol. In an example, a detectable compound less likely to be naturally occurring in the patient organism may be preferred. In an example, any organic compound generated over time based on the combination of reagent, catalyst, biological sample, and drive signal may be the detected compound.

In an example, biomeasurement engine 144 of reader 140 may be configured to execute to plot the fourth voltage (e.g. of measurement trace 254) as a sample voltage waveform, and the sample voltage waveform may be compared to one or more control waveforms (e.g., for the condition appropriate infectious agent) to determine whether the measured voltage waveform corresponds to a medical diagnosis. In an example, if the measured sample voltage waveform aligns with the control waveform, a medical diagnosis is made and reported via display 141. In another example, if the sample voltage curve or waveform fails to align with any control waveform, an error may be displayed on display 141. In an example, a complete failure to align with any waveform may be indicative of a defect in at least one of reagent vial 180, test strip 150, and reader 140. For example, one of the control wave forms may typically be a control for a non-infected patient, so a failure to align with a baseline control may indicate some form of contamination of the assay. In an example, control waveforms may cover a broader than optimal incubation period, so that slight timing errors in the incubation period (e.g., sample being deposited in test well 165 early or late) may be accounted for by biomeasurement engine 144. In an example, reader 140 reports a diagnosis state based on the concentration of the detectable compound (block 550). In an example, the diagnosis state is reported in a graphical report, and the graphical report abstracts numerical measurements of the fourth voltage and the concentration of the small molecule (e.g., paracetamol or glucose) as the detectable compound. For example, for ease of use and to guard against misinterpretation, rather than displaying any raw values for the detectable compound or microbe concentration, the significance of which may differ depending on the assay, a diagnosis may be computed by biomeasurement engine 144 and displayed unambiguously.

In an example, reader 140 may be configured to recalibrate with a second test strip and a second reagent on the second test strip, and after recalibrating, a second test sample is measured on the second test strip. In an example, the second test sample may be for a second patient for the same microbe as the first test sample. In another example, the second test sample may be for a different microbe for the same patient or a new patient. In an example, the second reagent may be a separate aliquot of the first reagent, kept sterile and biologically isolated from the first reagent. In an example, the second reagent may be in a second sealed reagent vial. In an example, a given test strip may have multiple test and/or calibration wells. In an example, each test well and calibration well pairing may be allocated to a different assay. In an example where multiple samples are tested for the same microbial infection, the multiple samples may be treated with a common stock of reagent that is calibrated once on a multi-test well test strip. For example, a common stock of reagent may be pipette into a plurality of reagent vials with different sample swabs and also onto the calibration well of the multi-well test strip. In the example, a second sample or test well of the multi-test well strip may measure the detectable compound concentration in a second sample biologically isolated from the first sample well. In an example, reader 140 may require a firmware and/or bios update to be reconfigured for a different assay for a different infectious microbe. In the example, such updates may be delivered wirelessly. In an example, a multi-well test strip may be configured to test for different infectious agents on different wells, potentially with separate calibration wells where necessary. For example, if two infectious diseases may be tested with the same reagent, but, for example, with different enzymes in different test wells, both may potentially be tested with the same sample-reagent mix from reagent vial 180.

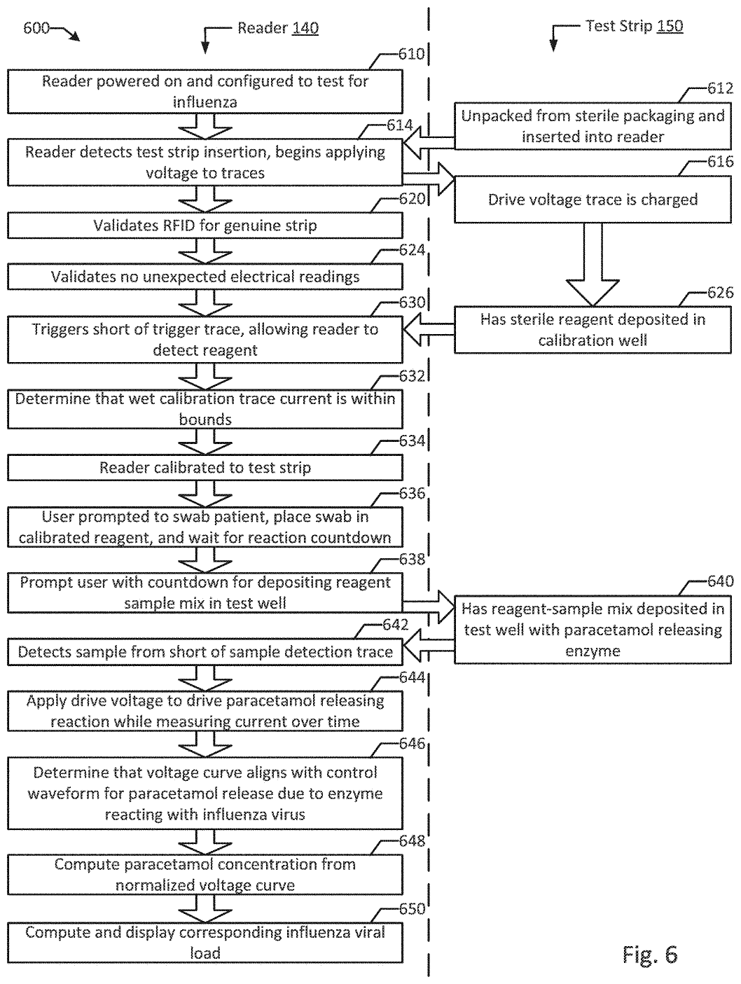

FIG. 6 is a flow diagram illustrating an example system employing the present portable microbial load detection according to an example of the present disclosure. Although the examples below are described with reference to the flowchart illustrated in FIG. 6, it will be appreciated that many other methods of performing the acts associated with FIG. 6 may be used. For example, the order of some of the blocks may be changed, certain blocks may be combined with other blocks, and some of the blocks described are optional. The methods may be performed by processing logic that may comprise hardware (circuitry, dedicated logic, etc.), software, or a combination of both. In example system 600, a reader 140 executes portable viral load detection on a sample on a test strip 150.

In an example, reader 140 is powered on and configured to test for influenza (block 610). In the example, test strip 150 is unpacked from sterile packaging and inserted into reader 140 (block 612). In an example, reader 140 detects test strip 150′s insertion, and begins applying the drive voltage to drive voltage traces 230 and 250 (block 614). In another example drive voltage may only be applied to drive voltage trace 230 to conserve power until the drive voltage is needed in the test well 165. In an example, drive voltage trace 230, and therefore calibration well 160 of test strip 150 is charged (block 616). In an example, reader 140 may validate an RFID on test strip 150 to ensure that test strip 150 is a genuine test strip (block 620). In an example, reader 140 may validate that there are no unexpected electrical readings from test strip 150 (e.g., unexpected shorts or other signals) (block 624). In an example, test strip 150 has sterile reagent deposited in calibration well 160 from reagent vial 180 (block 626). In another example, reagent vial 180 may have sample swab 190 inserted shortly prior to depositing reagent-sample mixture into calibration well 160. In an example, the reagent triggers a short of the trigger trace (e.g., reagent detection trace 232) allowing reader 140 to detect that reagent has been added to test strip 150 (block 630).

In an example, reader 140 determines whether a voltage plot over time of wet calibration trace 234's voltage measurements is within acceptable bounds (block 532). In an example, reader 140 is successfully calibrated to test strip 150 and reagent vial 180 (block 634). In an example, reader 140 prompts a user to swab a patient and to place the swab in calibrated reagent vial 180, and then to wait for a reaction countdown timer 244 shown on display 141 (block 636). In some examples, the swabbing of the patient may occur before calibration. In an example, reader 140 prompts the user with countdown timer 244 for depositing reagent-sample mixture into test well 165 (block 638). In an example, test strip 150 has a reagent-sample mix deposited in test well 165, which may be coated with a paracetamol (as the detectable compound) releasing enzyme (block 640). In an example, reader 140 may detect that a sample has been added to test strip 150 via a short between drive voltage trace 250 and sample detection trace 252 (block 642).

In an example, reader 140 may continue to apply drive voltage or a separate test voltage of a different magnitude to drive the paracetamol releasing reaction in test well 165, while measuring voltage and/or current on measurement trace 154 over time (block 644). In an example, drive voltage may be scaled, varied, and/or modulated based on a particular reaction in test well 165. In an example, biomeasurement engine 144 may determine that the voltage/amperage curve/waveform measured from measurement trace 154 for test strip 150 aligns with a control waveform for paracetamol release due to the enzyme reacting with influenza virus (block 646). In an example, biomeasurement engine 144 may compute the measured paracetamol concentration from a normalized voltage curve generated by comparing the raw measurement data (e.g., voltage waveform) from test strip 150 with known controls (block 648). In an example, reader 140, specifically biomeasurement engine 144 may compute and display a corresponding influenza viral load on display 141 (block 650).

In an example, the present portable microbial load detection may provide an alternative to nucleic acid based microbial load testing. By enabling precise calibration on a per test strip, per reagent vial basis, the present portable microbial load detection enables detection of reactions generating signals that would otherwise likely be considered noise in a typical reader. In an example, the present portable microbial load detection may be performed without cryogenic or chemical means for nucleic acid extraction from samples, and without centrifuges and thermocyclers which may severely impact transportability and availability for microbial load testing equipment. In an example, while sterility is still crucial, the present portable microbial load detection may have reduced likelihood of cross contamination as compared to nucleic acid based testing. Therefore, the present portable microbial load detection may be flexibly brought to the patient as needed enabling better diagnosis and treatment options.

In one embodiment, the present system and methods of using such a system is capable of detecting the detectable compound (thus the microbial load) at a low concentration, which a commercially available device (e.g., a commercially available glucose meter) is unable to detect. In one embodiment, the present system and methods of using such a system is capable of detecting the detectable compound (e.g., paracetamol) at a concentration of 1 mM or below 1 mM. In one embodiment, the present system and methods of using such a system is capable of detecting the detectable compound (e.g., paracetamol) at a concentration selected from the group consisting of 0.9 mM, 0.8mM, 0.7 mM, 0.6 mM, 0.5 mM, 0.4 mM, 0.5 mM, 0.4 mM, 0.3 mM, 0.2 Mm, 0.1 mM and 0.05 mM.

In one embodiment, the present system and methods of using such a system may be capable of detecting the detectable compound (e.g., paracetamol) at a concentration below 0.05 mM.

In one embodiment, the present system and methods of using such a system may be used to detect the detectable compound (e.g., paracetamol or glucose) at a concentration of about 0.01 mM.

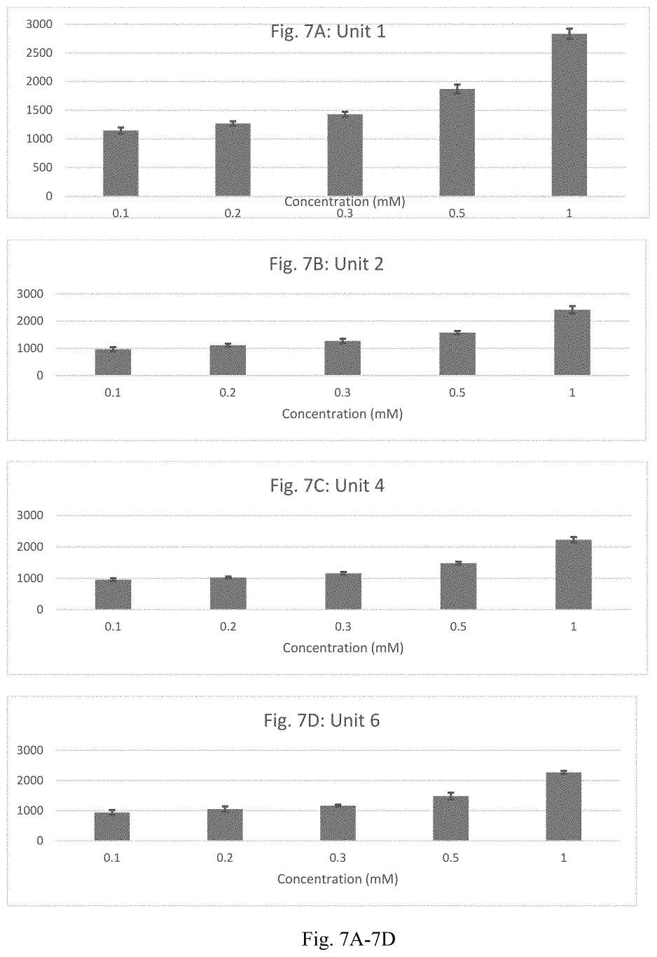

FIGS. 7A-7D demonstrated that the as-measured electrical signals (e.g., nanoamps) of the present device linearly correlate with different concentrations of a detectable compound (e.g., paracetomol; mMol) according to an example of the present disclosure.

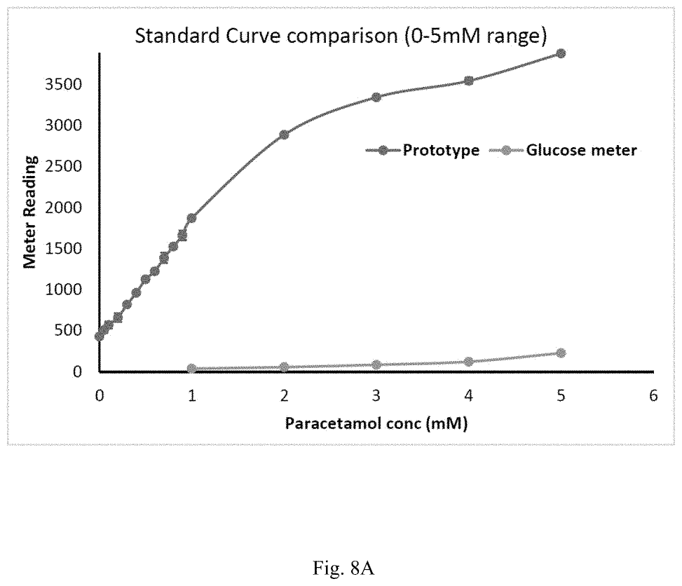

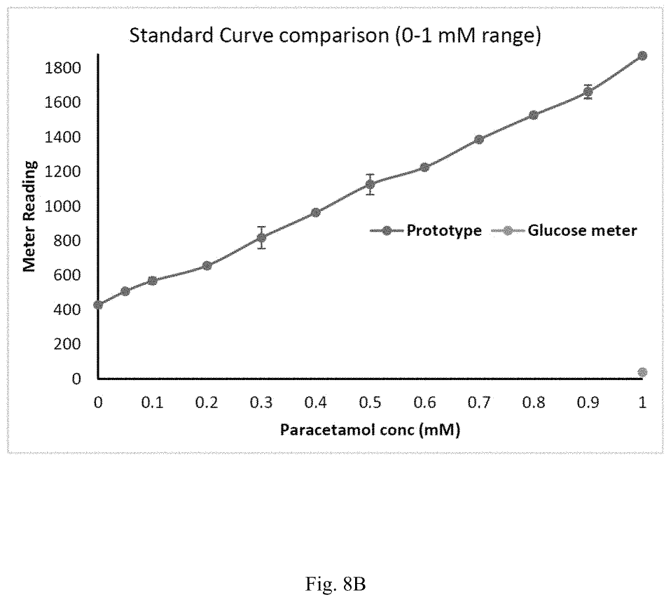

FIGS. 8A-8B showed that the present system demonstrated much higher sensitivity on detection of influenza virus as compared with a commercial glucose meter (One Touch Ultra 2 meter; www.onetouch.com/products/glucose-meters/onetouch-ultra2).

It will be appreciated that all of the disclosed methods and procedures described herein can be implemented using one or more computer programs or components. These components may be provided as a series of computer instructions on any conventional computer readable medium or machine readable medium, including volatile or non-volatile memory, such as RAM, ROM, flash memory, magnetic or optical disks, optical memory, or other storage media. The instructions may be provided as software or firmware, and/or may be implemented in whole or in part in hardware components such as ASICs, FPGAs, DSPs or any other similar devices. The instructions may be executed by one or more processors, which when executing the series of computer instructions, performs or facilitates the performance of all or part of the disclosed methods and procedures.

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims.

### EXAMPLES

### Example 1

Applicant has developed a novel electrochemical assay that uses a biosensor or the system disclosed herein to detect influenza virus or other microbial. The present system uses a specific enzyme present on the surface of an influenza virus. The present system uses a chemical compound as a precursor compound after being exposed to the patient samples. See Scheme 1. The target enzyme, if present, will cleave the precursor compound and release an electrochemically active molecule as the detectable compound, which can be detected amperometrically by using the present system. Therefore, enzyme specific to influenza infection can be rapidly detected and quantified. The present system provides a certain voltage, which triggers the paracetamol to release electrons. See Scheme 2. These electrons are then measured by using the present system.

The clinically positive influenza sample contains an enzyme which can cleave the precursor compound (e.g., SP(OMe)₂) and release paracetamol. A certain voltage provided by the present system can trigger the release of electrons from the paracetamol. The released electrons are measured by the present system.

In one embodiment, the present system and methods of using such a system may be expanded to include any other viruses or pathogen. For example, one could design specific chemical compounds as precursor compounds for each pathogen/virus. The presence of that specific pathogen/virus will trigger cleavage of the precursor compounds to release a detectable compound and the release of electrons from that the detectable compound and one can detect the signal output by using the present system.

In one embodiment, one can provide detection kits specific to each virus or pathogen. For each pathogen/virus, the kits will be labelled clearly and specifically. An ordinary person would purchase the kit for one pathogen and follow the steps to obtain the results.

For example, for detection of E. coli, one can use the specific chemical compound GP as the precursor compound, that can be cleaved by an enzyme (beta-galactosidase) produced by E. coli. The released paracetamol can be detected by the present system. The detailed synthesis of GP was described in Anal. Chem. 2018, 90, 19, 11589-11598.

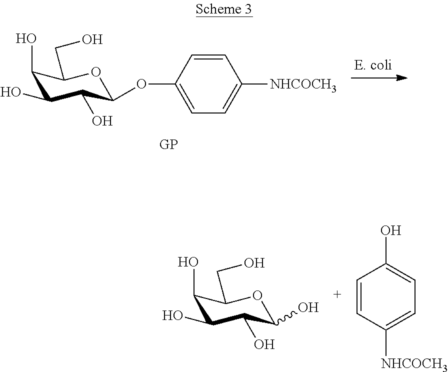

In one embodiment, the present system can successfully detect influenza virus, present in the nasopharyngeal swab of the patient (See Scheme 2). In one embodiment, the present system can provide two readings: the first reading at 0 minute to determine the baseline value (the baseline value detects a known concentration of paracetamol), which is the output (this output value records the known concentration of paracetamol plus the excess concentration of paracetamol that was released due to the presence of the virus) and the second reading at 15 minutes. The value obtained from subtracting 0 minute readout from the 15 minute readout will indicate either presence or absence of influenza virus in the sample. For example, if the output value remains same as the baseline value, then clinical sample does not contain the Influenza virus. If the output value is higher than the baseline value, then clinical sample does contain the Influenza virus.

### Example 2

The present system demonstrated much higher sensitivity on detection of influenza virus as compared with a commercial glucose meter.

Purpose: to test nine throat swab samples purchased from the sample bank and two cultured virus samples by using the present system and a store-bought glucose meter (One Touch Ultra 2 meter; www.onetouch.com/products/glucose-meters/onetouch-ultra2).

Throat swab samples (TS): The throat swab samples were purchased from a sample bank and the study was performed under double blind conditions. The person running the assay was unaware of the specimen characteristics until they finished all testing and analysis.

Cultured virus samples (CVS): The virus (i.e., influenza A) was infected in MDCK cell lines and then grown over a period of 2-3 days and then tested by q RT-PCR and Plaque assays.

TABLE 1
CVS Samples (CVS-1 and CVS-2 are
two different strains of Influenza A).

| Sample | Specimen characteristics | Virus titer |
|---|---|---|
| CVS-1 | A/California/12/2012 | 10⁵ pfu/ml |
| CVS-2 | A/Aichi/2/1968 | 10⁷ pfu/ml |

TABLE 2
Standard curve for paracetamol.

| | | Sensor reading | | | | | | | |
|---|---|---|---|---|---|---|---|---|---|
| | | Prototype | | | | Glucose meter | | | |
| Sr | Parecetamol | readout | | | | readout | | | |
| No. | (mM) | 1 | 2 | AVG | RSD | 1 | 2 | AVG | RSD |
| 1 | 5 | 3876 | 3878 | 3877 | 0.0 | 220 | 232 | 226 | 6.4 |
| 2 | 4 | 3571 | 3516 | 3544 | 1.1 | 112 | 132 | 122 | 14.1 |
| 3 | 3 | 3331 | 3356 | 3344 | 0.5 | 80 | 89 | 84.5 | 6.3 |
| 4 | 2 | 2884 | 2887 | 2886 | 0.1 | 55 | 57 | 56 | 1.4 |
| 5 | 1 | 1867 | 1875 | 1871 | 0.3 | 37 | 39 | 38 | 1.4 |

| | | | | | | | | |
|---|---|---|---|---|---|---|---|---|
| 6 | 0.9 | 1621 | 1704 | 1663 | 3.5 | low | low | NA |
| 7 | 0.8 | 1526 | 1527 | 1527 | 0.0 | low | low | NA |
| 8 | 0.7 | 1341 | 1431 | 1386 | 4.6 | low | low | NA |
| 9 | 0.6 | 1235 | 1214 | 1225 | 1.2 | low | low | NA |
| 10 | 0.5 | 1139 | 1113 | 1126 | 1.6 | low | low | NA |
| 11 | 0.4 | 973 | 953 | 963 | 1.5 | low | low | NA |
| 12 | 0.3 | 805 | 831 | 818 | 2.2 | low | low | NA |
| 13 | 0.2 | 693 | 618 | 656 | 8.1 | low | low | NA |
| 14 | 0.1 | 536 | 599 | 568 | 7.8 | low | low | NA |
| 15 | 0.05 | 531 | 483 | 507 | 6.7 | low | low | NA |
| 16 | 0 | 403 | 453 | 428 | 8.3 | low | low | NA |

* low as a glucose meter readout means that the glucose meter is unable to detect any paracetamol in the sample solution.

FIGS. 8A and 8B showed the comparative results between a standard curve of paracetamol using both a prototype meter according to an example of the present disclosure and a commercially available glucose meter (One Touch Ultra 2 meter; www.onetouch.com/products/glucose-meters/onetouch-ultra2).

For example, FIG. 8A is a graph illustrating comparative results between a standard curve of paracetamol using both a prototype meter according to an example of the present disclosure and a commercially available glucose meter. The standard curve in the range (0-5 mM): The prototype meter according to an example of the present disclosure can detect in this entire range (0-5 mM), however the commercially available glucose meter can detect the presence of the Paracetamol only in 1-5 mM range.

For example, FIG. 8B is a graph illustrating another set of comparative results between a standard curve of paracetamol using both a prototype meter according to an example of the present disclosure and a commercially available glucose meter. The standard curve in the range (0-1 mM): The prototype meter according to an example of the present disclosure can detect in this range (0-1 mM) of the of paracetamol and show a linear co-relation, however the commercially available glucose meter can only detect 1 mM of the paracetamol, and does not show any meaningful reading for any of the concentration below 1 mM for paracetamol (0-0.9 mM range).

Detection of Throat Swab and Cultured Virus Samples:

We tested 9 throat swab samples (5 positive Influenza virus sample & 4 negative virus sample) with the proprietary compound SP(OMe)2 and detected the virus using both the present system and the store-bought glucose meter (One Touch Ultra 2 meter; www onetouch.com/products/glucose-meters/onetouch-ultra2).

Scheme 4 shows rapid detection of Influenza virus using the present system or the commercial glucose meter (One Touch Ultra 2 meter; www.onetouch.com/products/glucose-meters/onetouch-ultra2).

Test conditions: 50 μl of Throat swab (TS)/cultured virus sample (CVS)+30 μl of Lysis buffer (PBS. pH-6.5, 1% triton-X100 and 20 mM CaC₂, +10 l 1.5 mM paracetamol (final conc=0.15 mM/100 μl)+10 μl of 10 mM SP(OMe)2 (final conc=1 mM/100 μl); Total test volume=100 μl.

Controls:

Positive controls:

1. 1.5 mM Paracetamol solution (100 μl)

2. 50 μl pure A/Anhui/ Neuraminidase (NA) (50U)+30 μl of Lysis buffer (PBS. pH-6.5, 1% triton-X100 and 20 mM CaCl_{2,} +10 μl 1.5 mM paracetamol (final conc=0.15 mM/100+10 μl of 10 mM SP(OMe)₂ (final conc=1 mM/100 μl); Total test volume=100 μl.

Negative controls:

3. 50 μl throat swab+30 μl of Lysis buffer (PBS. pH-6.5, 1% triton-X100 and 20 mM CaCl₂, +10 μl 1.5 mM paracetamol (final conc=0.15 mM/100 μl)+10 μl of DI water. Total test volume=100 μl

4. 50 μl DI water+30 μl of Lysis buffer (PBS. pH-6.5, 1% triton-X100 and 20 mM CaCl₂, +10 μl 1.5 mM paracetamol (final conc=0.15 mM/100 μl)+10 μl of 10 mM SP(OMe)₂ (final conc=1 mM/100 μl); Total test volume=100 μl

5. Lysis buffer (100 μl)

6. 50 μl of cultured virus sample+30 μl of Lysis buffer (PBS. pH-6.5, 1% triton-X100 and 20 mM CaC₂, +10 μl 1.5 mM paracetamol (final conc=0.15 mM/100+10 μl of DI water. Total test volume=100 μl

7. Distilled water (100 μl).

TABLE 3
Test results.

| | | | Sensor Readings | | | | | | | | | |
|---|---|---|---|---|---|---|---|---|---|---|---|---|
| | | | Prototype meter | | | | | Glucose meter | | | | |
| | | | | | | | Accurate | | | | | Accurate |
| Sr No. | Sample ID | Sample Characteristics | t = 0 mins | | t = 30 mins | | Dectection | t = 0 mins | | t = 30 mins | | Detection |
| 1 | TS-1 | influenza A positive | 774 | 769 | 1694 | 1684 | Yes | Low | Low | Low | Low | No |
| 2 | TS-2 | influenza A positive | 569 | 589 | 1805 | 1782 | Yes | Low | Low | Low | Low | No |
| 3 | TS-3 | influenza A positive | 672 | 684 | 1875 | 1896 | Yes | Low | Low | 36 | 37 | Yes |
| 4 | TS-4 | influenza A positive | 699 | 672 | 1678 | 1576 | Yes | Low | Low | Low | Low | No |
| 5 | TS-5 | influenza A positive | 670 | 654 | 1839 | 1822 | Yes | Low | Low | Low | Low | No |
| 6 | TS-6 | influenza A negative | 542 | 561 | 543 | 569 | Yes | Low | Low | Low | Low | No |
| 7 | TS-7 | influenza A negative | 664 | 656 | 681 | 672 | Yes | Low | Low | Low | Low | No |
| 8 | TS-8 | influenza A negative | 564 | 558 | 547 | 599 | Yes | Low | Low | Low | Low | No |
| 9 | TS-9 | influenza A negative | 494 | 535 | 545 | 611 | Yes | Low | Low | Low | Low | No |
| 10 | Control-1 | Positive control | 2654 | 2564 | 2564 | 2455 | Yes | 40 | 43 | 47 | 55 | Yes |
| | | 1.5 mM Paracetamol | | | | | | | | | | |
| 11 | Control-2 | Positive control | 889 | 901 | 2613 | 2658 | Yes | 51 | 48 | 45 | 49 | Yes |
| | | (Pure A/Anhul/NA (500)) | | | | | | | | | | |
| 12 | Control-3 | Negative control | 630 | 642 | 654 | 623 | Yes | Low | Low | Low | Low | Yes |
| | | (Throat swab) | | | | | | | | | | |
| 13 | Control-4 | Negative control | 467 | 478 | 465 | 467 | Yes | Low | Low | Low | Low | Yes |
| | | (SP(OMe)₂) | | | | | | | | | | |
| 14 | Control-5 | Negative control | 488 | 478 | 454 | 532 | Yes | Low | Low | Low | Low | Yes |
| | | (Lysis Buffer) | | | | | | | | | | |
| 15 | Control-6 | Negative control | 663 | 673 | 643 | 654 | Yes | Low | Low | Low | Low | Yes |
| | | (Cultured virus sample) | | | | | | | | | | |
| 16 | Control-7 | Negative control | 436 | 465 | 434 | 432 | Yes | Low | Low | Low | Low | Yes |
| | | (Distilled water) | | | | | | | | | | |
| 17 | CVS-1 | A/California/12/2012 | 869 | 873 | 1675 | 1715 | Yes | Low | Low | Low | Low | No |
| 18 | CVS-2 | A/Aichi/2/1988 | 879 | 914 | 1878 | 1869 | Yes | Low | Low | 35 | 38 | Yes |

* low as a glucose meter readout means that the glucose meter is unable to detect any paracetamol in the sample solution.

We tested nine throat swab samples and two cultured virus samples by using the present system and a store-bought glucose meter. For example, at t=0 minute, the present system detected 654-774 nanoamps for all the influenza A positive samples of TS-1 to TS-5. At t=30 minutes however, the present system detected 1576-1896 nanoamps for all the influenza A positive samples of TS-1 to TS-5, confirming the presence of the influenza A virus. As comparison, the present system did not detect any significantly difference between t=0 and t=30 for influenza A negative samples (TS-6 to TS-9), for controls (Control-1 to Control-7). Similarly, the present system detected 869-914 nanoamps for both CVS samples of CVS-1 to CVS-2 at t=0 minute. At t=30 minutes however, the present system detected 1675-1878 nanoamps for both CVS samples of CVS-1 to CVS-2, confirming the presence of the influenza A virus.

We were able to detect all the samples accurately using the present system, but the glucose meter was able to detect just one throat swab sample and one cultured virus sample correctly. Thus, the present system is highly sensitive and can accurately detect low titers of the influenza virus.

The invention is claimed as follows:

1. A system of detecting a concentration of a detectable compound, the system comprising:
a first test strip including a calibration well and at least a first sample well, wherein the calibration well is associated with at least a first calibration trace, a second calibration trace, and a third calibration trace, and the first sample well is associated with at least a first test trace, a second test trace, and a third test trace, wherein at least one of the first sample well, the first test trace, the second test trace, and the third test trace comprises a precursor compound;
a reader including one or more processors and an amplifier, the one or more processors configured to execute to:
detect a connection with the first test strip;
apply a drive signal to the first test strip, wherein the drive signal is applied to the first calibration trace and the first test trace;
measure a first electrical signal of the second calibration trace, wherein the first electrical signal indicates that a first reagent comprising a first concentration of the detectable compound has been added to the calibration well;
measure a second electrical signal of the third calibration trace over a first calibration time period, wherein the third calibration trace is associated with the first reagent comprising the first concentration of the detectable compound;
calibrate the reader to the first reagent on the first test strip based on the second electrical signal;
detect an addition of a first test sample in the first sample well based on measuring a third electrical signal on the second test trace, wherein the first test sample comprises the first reagent, and wherein an incubation period elapses before the first test sample is added to the first sample well, and a first biological sample is added into the first reagent during the incubation period and the first biological sample comprises an enzyme, wherein the enzyme catalyzes a reaction of the precursor compound to produce a second concentration of the detectable compound;
measure a fourth electrical signal of the third test trace over a first test time period;
calculate the second concentration of the detectable compound in the first test sample; and
report a diagnosis state based on the concentration of the detectable compound.

2. The system of claim 1, wherein the electrical signal is voltage.

3. The system of claim 1, wherein the detectable compound is either glucose or paracetamol.

4. The system of claim 1, wherein insertion of the first test strip is based on detecting a transient electrical pulse after the insertion of the first test strip, and the first test strip is validated after detecting the transient electrical pulse.

5. The system of claim 1, wherein the one or more processors further executes to:
plot the fourth electrical signal over the first test time period as a first electrical signal waveform;
determine whether the first electrical signal waveform aligns with a control waveform associated with a medical diagnosis;
responsive to determining that the first electrical signal waveform aligns with the control waveform, report the medical diagnosis; and
responsive to determining that the first electrical signal waveform fails to align with the control waveform, display an error on the reader.

6. The system of claim 1, wherein the amplifier is a transimpedance amplifier.

7. The system of claim 6, wherein each of the first calibration trace, the second calibration trace, the third calibration trace, the first test trace, the second test trace, and the third test trace is connected to a separate channel in the transimpedance amplifier.

8. The system of claim 6, wherein at least two signals corresponding respectively to at least two of the first calibration trace, the second calibration trace, the third calibration trace, the first test trace, the second test trace, and the third test trace are multiplexed together in the transimpedance amplifier.

9. The system of claim 5, wherein the transimpedance amplifier in a first configuration mode measures a first signal type associated with a first tested medical condition associated with the first biological sample, and the transimpedance amplifier in a second configuration mode measures a second signal type associated with a second tested medical condition associated with a second biological sample.

10. The system of claim 1, wherein the reader is configured to recalibrate with a second test strip and a second reagent comprising a first concentration of the detectable compound on the second test strip, and after recalibrating, a second test sample comprising the second reagent is measured on the second test strip, wherein an incubation period elapses before the second test sample is added to a second sample well, and a second biological sample is added into the second reagent during the incubation period and the second biological sample comprises the enzyme, wherein the enzyme catalyzes the reaction of the precursor compound to produce a third concentration of the detectable compound.

11. The system of claim 10, wherein the second reagent is a separate aliquot of the first reagent.

12. The system of claim 11, wherein the first reagent and the second reagent are individually biologically isolated.

13. The system of claim 1, wherein the first test strip includes a second sample well biologically isolated from the first sample well, and the second sample well is used to measure a second test sample.

14. The system of claim 1, wherein the diagnosis state is reported in a graphical report, and the graphical report abstracts numerical measurements of the fourth electrical signal and the second concentration of the detectable compound.

15. The system of claim 1, wherein the detectable compound is paracetamol.

16. The system of claim 1, wherein the second concentration of the detectable compound is either 1 mM or below 1 mM.

17. The system of claim 1, wherein the second concentration of the detectable compound is a concentration selected from the group consisting of 0.9 mM, 0.8mM, 0.7 mM, 0.6 mM, 0.5 mM, 0.4 mM, 0.5 mM, 0.4 mM, 0.3 mM, 0.2 Mm, 0.1 mM and 0.05 mM.

18. A method of detecting a concentration of a detectable compound, the method comprising:
detecting a connection with a test strip, wherein the test strip includes a calibration well associated with at least a first calibration trace, a second calibration trace, and a third calibration trace and at least a first sample well associated with at least a first test trace, a second test trace, and a third test trace, wherein at least one of the first sample well, the first test trace, the second test trace, and the third test trace comprises a precursor compound;
applying a drive signal to the test strip, wherein the drive signal is applied to the first calibration trace and the first test trace;
measuring a first electrical signal of the second calibration trace, wherein the first electrical signal indicates that a first reagent comprising a first concentration of the detectable compound has been added to the calibration well;
measuring a second electrical signal of the third calibration trace over a first calibration time period, wherein the third calibration trace is associated with the first reagent comprising a first concentration of the detectable compound;
calibrating the reader to the first reagent on the first test strip based on the second electrical signal;
detecting an addition of a first test sample in the first sample well based on measuring a third electrical signal on the second test trace, wherein the first test sample comprises the first reagent, and wherein an incubation period elapses before the first test sample is added to the first sample well, and a first biological sample is added into the first reagent during the incubation period and the first biological sample comprises an enzyme, wherein the enzyme catalyzes a reaction of the precursor compound to produce a second concentration of the detectable compound;
measuring a fourth electrical signal of the third test trace over a first test time period;
calculating a the second concentration of the detectable compound in the first test sample; and
reporting a diagnosis state based on the concentration of the detectable compound.

19. The method of claim 18, wherein the electrical signal is voltage.

20. The method of claim 18, wherein the detectable compound is either glucose or paracetamol.

21. The method of claim 18, wherein the detectable compound is paracetamol.

22. The method of claim 18, wherein the second concentration of the detectable compound is 1 mM or below 1 mM.

23. The method of claim 18, wherein the second concentration of the detectable compound is a concentration selected from the group consisting of 0.9 mM, 0.8mM, 0.7 mM, 0.6 mM, 0.5 mM, 0.4 mM, 0.5 mM, 0.4 mM, 0.3 mM, 0.2 Mm, 0.1 mM and 0.05 mM.

24. A non-transitory computer readable storage medium storing one or more computer programs adapted to cause a processor based system to execute steps comprising:
detecting a connection with a test strip, wherein the test strip includes a calibration well associated with at least a first calibration trace, a second calibration trace, and a third calibration trace and at least a first sample well associated with at least a first test trace, a second test trace, and a third test trace, wherein at least one of the first sample well, the first test trace, the second test trace, and the third test trace comprises a precursor compound;
applying a drive signal to the test strip, wherein the drive signal is applied to the first calibration trace and the first test trace;
measuring a first electrical signal of the second calibration trace, wherein the first voltage indicates that a first reagent comprising a first concentration of a detectable compound has been added to the calibration well;
measuring a second electrical signal of the third calibration trace over a first calibration time period, wherein the third calibration trace is associated with the first reagent comprising the first concentration of the detectable compound;
calibrating the reader to the first reagent on the first test strip based on the second electrical signal;
detecting an addition of a first test sample in the first sample well based on measuring a third electrical signal on the second test trace, wherein the first test sample comprises the first reagent, and wherein an incubation period elapses before the first test sample is added to the first sample well, and a first biological sample is added into the first reagent during the incubation period and the first biological sample comprises an enzyme, wherein the enzyme catalyzes a reaction of the precursor compound to produce a second concentration of the detectable compound;
measuring a fourth electrical signal of the third test trace over a first test time period;
calculating the second concentration of the detectable compound in the first test sample; and
reporting a diagnosis state based on the second concentration of the detectable compound.

25. The non-transitory computer readable storage medium of claim 24, wherein the electrical signal is voltage.

26. The non-transitory computer readable storage medium of claim 24, wherein the detectable compound is either glucose or paracetamol.

27. The non-transitory computer readable storage medium of claim 24, wherein the detectable compound is paracetamol.

28. The non-transitory computer readable storage medium of claim 24, wherein the second concentration of the detectable compound is either 1 mM or below 1 mM.

29. The non-transitory computer readable storage medium of claim 24, wherein the second concentration of the detectable compound is a concentration selected from the group consisting of 0.9 mM, 0.8 mM, 0.7 mM, 0.6 mM, 0.5 mM, 0.4 mM, 0.5 mM, 0.4 mM, 0.3 mM, 0.2 Mm, 0.1 mM and 0.05 mM.
